(19) United States
(12) Patent Application Publication
(10) Pub. No.: US 2026/0174452 A1
(43) Pub. Date: Jun. 25, 2026

(54) SHOCK WAVE BALLOON DILATION CATHETER AND SHOCK WAVE BALLOON DILATION CATHETER FOR THE TRICUSPID VALVE OPENING

(71) Applicant: VASCUPATENT MEDICAL (SHENZHEN) CO., LTD., Shenzhen, Guangdong (CN)

(72) Inventors: Lizhong LU, Shenzhen, Guangdong (CN); Chen CHEN, Shenzhen, Guangdong (CN); Junxiong OUYANG, Shenzhen, Guangdong (CN); Lili WANG, Shenzhen, Guangdong (CN)

(21) Appl. No.: 18/850,579

(22) PCT Filed: May 13, 2024
(86) PCT No.: PCT/CN2024/092831

§ 371 Date: Sep. 25, 2024

(30) Foreign Application Priority Data
May 17, 2023 (CN) 202310557947.1
Jul. 7, 2023 (CN) 202310828225.5
Jul. 7, 2023 (CN) 202310833357.7
Jul. 7, 2023 (CN) 202310833385.9
May 13, 2024 (WO) PCT/CN2024/092831

(51) Int. Cl. A61B 17/22; A61F 2/24; A61M 25/10

(52) U.S. Cl. CPC A61B 17/2202; A61B 17/22022; A61F 2/2418; A61M 25/10; A61B 2017/22061

(57) ABSTRACT

The present invention discloses a shock wave balloon dilation catheter comprising a balloon and a dilation catheter. The balloon includes a blood flow passage on its outer circumference and/or interior, extending axially and penetrating through the outer circumference and/or center. A shock wave generator is arranged within the balloon chamber, where conductive fluid can be introduced. The distal end of the dilation catheter passes through or into the balloon and is sealedly connected to the balloon. A fluid passage in the catheter communicates with the balloon chamber, and the shock wave generator is fixed to the portion of the catheter within the balloon. The dilation catheter is provided with a guide wire lumen. The present invention further discloses a shock wave balloon dilation catheter for tricuspid valve openings, which designed to avoid blood flow blockage and reduce the hemodynamic impact during the procedure, enhancing safety and treatment effectiveness.

### BACKGROUND OF THE PRESENT INVENTION

### Field of Invention

The present invention relates to a medical device, and particularly relates to a shock wave balloon dilation catheter and a shock wave balloon dilation catheter for the tricuspid valve opening.

### Description of Related Arts

A heart valve is a fundamental structure of the heart and may suffer from incomplete closure (regurgitation) or stenosis due to congenital or acquired inflammatory factors, affecting the quality of life of the patient and, in severe cases, threatening life. With the intensification of aging in China, the incidence of degenerative heart valve diseases is expected to rise, leading to left ventricular outflow tract obstruction in the end stages of the disease, resulting in reduced cardiac output, decreased exercise capability, heart failure, and death due to cardiovascular causes. The incidence in the 50-59 age group is approximately 0.2%, while in the 80-90 age group, the incidence rises to 9.8%.

Wherein, the tricuspid valve is a common area for heart valve diseases. When calcification occurs in the tricuspid valve, it can prevent the valve from closing properly. This can lead to gaps in the tricuspid valve, causing upper atrium pressure leakage and preventing blood pressure from reaching the desired vascular pressure. To address this issue, a common solution is the implantation of an artificial valve, but before implanting the artificial valve, the tricuspid valve needs to be dilated using a balloon so that the artificial valve can be implanted. However, during balloon dilation, there is a risk of temporary vascular blockage. If not handled correctly, this procedure can pose a risk of fatality.

Traditional balloon valvuloplasty offers only modest hemodynamic improvements, and has a high incidence of restenosis. The long-term survival rate post-surgery does not show significant differences compared to the natural history of valve stenosis. Currently, the focus of treating valve stenosis has shifted from balloon valvuloplasty to transcatheter valve replacement, but several issues remain, including valve durability, preservation of coronary artery pathways, and leaflet thrombosis. Balloon dilation is a critical step in the procedure, and improving the effectiveness of balloon dilation for valve treatment has become our research focus.

### SUMMARY OF THE PRESENT INVENTION

The objective of the present invention is to provide a valve shock wave balloon dilation catheter, and the technical problem to be solved is to treat calcification of the heart valve while maintaining a certain level of blood flow during the treatment process, ensuring the safety of the surgery and improving treatment efficiency.

To address the aforementioned issues, the present invention adopts the following technical solution: a shock wave balloon dilation catheter, which sequentially comprises, from the distal end to the proximal end, a balloon and a dilation catheter.

The balloon is provided with a blood flow passage on the outer circumference and/or inside of the balloon, the blood flow passage extends axially along the balloon and penetrates through the outer circumference and/or the center of the balloon, a shock wave generator is provided in the chamber of the balloon, and conductive fluid can be introduced into the chamber of the balloon;

The distal end of the dilation catheter either passes through the balloon or enters the balloon. The dilation catheter is sealedly connected to the balloon, a fluid passage is provided on the dilation catheter that communicates with the chamber of the balloon. The shock wave generator is fixed to the portion of the dilation catheter that is positioned within the chamber of the balloon, and the dilation catheter is provided with a guide wire lumen.

Furthermore, the balloon is composed of at least three balloon bodies, supporting rods are provided at both the proximal end and the distal end of the balloon bodies, respectively. The support rods at the proximal end of the balloon bodies are formed by branches from the wall of the dilation catheter. The distal end of the balloon body is a sealed surface. The support rods at the proximal end and the distal end of the balloon bodies are fixedly connected to the dilation catheter, respectively. The dilation catheter passes through the center of the balloon. The shock wave generator is provided in the chamber of at least two of the balloon bodies, and the balloon body with the shock wave generator is provided with fluid-conducting tube. The shock wave generator is fixed onto the fluid-conducting tube, the distal end of the fluid-conducting tube is fixed to the distal end of the chamber of the balloon body, and the proximal end of the fluid-conducting tube is fixed to the proximal end of the chamber of the balloon body. The support rod at the proximal end is provided with support rod cavity that communicates with the chamber of the balloon body and the fluid passage, the proximal end of the fluid-conducting tube in the balloon body with the shock wave generator communicates with the support rod cavity, and the fluid-conducting tube is provided with through-holes.

Furthermore, the number of the balloon bodies is a multiple of 2.

Furthermore, the outer wall of the balloon is provided with at least three arcuate protrusions distributed circumferentially along the balloon. The inner cavity of the arcuate protrusions communicates with the inner cavity of the balloon, forming an integral structure. An arcuate recess is provided between the two adjacent arcuate protrusions, forming a blood flow passage. The distal end of the dilation catheter passes through the chamber of the balloon, while the proximal end and the distal end of the balloon are sealedly connected to the dilation catheter.

Furthermore, reinforcing ribs are provided on the arcuate recesses, the reinforcing ribs are arranged axially along the balloon.

Furthermore, the shock wave generator comprises a plurality of electrode rings, insulating sleeves, and metal sleeves. The number of the insulating sleeves is the same as the number of the electrode rings. The dilation catheter is provided with conducting wires, the conducting wires are provided with exposed copper sections. The metal sleeves are fitted over the exposed copper sections of the conducting wires, the insulating sleeves are arranged outside of the metal sleeves to cover the metal sleeves. The electrode rings are fitted over the insulating sleeves, at least one electrode ring discharge through-hole is arranged on the electrode rings, and the electrode ring discharge through-holes are oriented toward the outer circumference of the balloon. The insulating sleeve through-holes that expose the metal sleeves are provided at the location where the insulating sleeves is located in the metal sleeves.

Furthermore, the two adjacent electrode ring discharge through-holes on the electrode rings are arranged in a staggered manner.

Furthermore, the electrode rings consist of two symmetrically arranged ring bodies, a connecting portion is provided between the two ring bodies, and the two ring bodies are connected by the connecting portion. The electrode ring discharge through-holes are respectively arranged on the two ring bodies.

Furthermore, a plurality of the electrode rings are connected in series or parallel by the conducting wires.

Furthermore, when a plurality of the electrode rings are connected in series by the conducting wires, the conducting wires comprise at least one positive wires and a common negative wire. The positive wires are provided with exposed copper sections at the electrode ring discharge through-holes of each of the electrode rings; or

The conducting wires further comprise a connecting wire, the exposed copper section of the positive wire is aligned with the electrode ring discharge through-hole of the electrode ring of the nearest end. Both ends of the connecting wire are provided with exposed copper sections. The exposed copper sections of the connecting wire are aligned with the electrode ring discharge through-holes of one of the adjacent electrode rings.

Furthermore, when a plurality of the electrode rings are connected in parallel by the conducting wires, the conducting wires comprise positive wires and negative wires that match the number of the electrode rings. The exposed copper section of each of the positive wires corresponds to one of the electrode ring discharge through-hole of one of the electrode ring, and the exposed copper section of each of the negative wires corresponds to another discharge through-hole of the same electrode ring.

Furthermore, the shock wave generator comprises at least one pair of electrode plates, a gap is arranged between each pair of the electrode plates. The dilation catheter is provided with the conducting wires, the conducting wires are electrically connected to the electrode plates.

Furthermore, the electrode plates are sheet-shaped.

Furthermore, each pair of the electrode plates is connected in series or parallel by the conducting wires.

Furthermore, when each of the electrode plates pairs is connected in series by the conducting wires, the conducting wires comprise a positive wire, a connecting wire and a negative wire, one electrode plate in a pair is electrically connected to the positive wire, the other electrode plate in the same pair is electrically connected to one electrode plate in another pair through the connecting wire, and the remaining electrode plate in that second pair is electrically connected to the negative wire.

Furthermore, when each of the electrode plates pairs is connected in parallel by the conducting wires, at least two sets of the conducting wires are provided, with each set comprising a positive wire and a negative wire, one pair of the electrode plates is electrically connected to one set of the positive wire and the negative wire, respectively, while another pair of the electrode plates is electrically connected to another set of the positive wire and the negative wire, respectively, forming a parallel connection.

The present invention further discloses a shock wave balloon dilation catheter for the tricuspid valve opening, comprising the shock wave balloon dilation catheter.

Compared with the prior art, the present invention provides a blood flow passage in the outer circumference and/or inside of the balloon and arranges a shock wave generator within the chamber of the balloon. After the balloon is inflated, the calcified portions are shattered by the shock wave generator. The blood flow passage ensures that a certain amount of blood flow passes during treatment, avoiding complete blockage of blood flow channel and reducing the hemodynamic impact of the balloon dilation procedure, thereby ensuring the safety of the procedure and improving treatment outcomes.

### BRIEF DESCRIPTION OF THE DRAWINGS

FIG. 1 is a schematic view of the overall structure of preferred embodiment 1.

FIG. 2 is a schematic structural view of the balloon in preferred embodiment 1.

FIG. 3 is a schematic view of the interior of the balloon in preferred embodiment 1.

FIG. 4 is a schematic structural view of the shock wave generator in preferred embodiment 1.

FIG. 5 is a schematic structural view of the interior of the shock wave generator in preferred embodiment 1.

FIG. 6 is a schematic view of the overall structure of preferred embodiment 2.

FIG. 7 is a schematic structural view of the interior of the balloon in preferred embodiment 2.

FIG. 8 is a left view of FIG. 7.

FIG. 9 is a schematic structural view of the shock wave generator in preferred embodiment 2.

FIG. 10 is a schematic structural view of the interior of the shock wave generator in preferred embodiment 2.

FIG. 11 is a schematic view of the overall structure of preferred embodiment 3.

FIG. 12 is a schematic structural view of the interior of the balloon in preferred embodiment 3.

FIG. 13 is a left view of FIG. 12.

FIG. 14 is a schematic structural view of the shock wave generator in preferred embodiment 3.

FIG. 15 is a schematic structural view of the interior of the shock wave generator in preferred embodiment 3.

FIG. 16 is a schematic view of the series connection of the shock wave generator in preferred embodiment 3.

FIG. 17 is a schematic view of the parallel connection of the shock wave generator in preferred embodiment 3.

FIG. 18 is a schematic view of the overall structure of preferred embodiment 4.

FIG. 19 is a schematic structural view of the balloon in preferred embodiment 4.

FIG. 20 is a schematic structural view of the shock wave generator in preferred embodiment 4.

FIG. 21 is a schematic structural view of the interior of the shock wave generator in preferred embodiment 4.

FIG. 22 is an enlarged partial view of FIG. 21.

### DETAILED DESCRIPTION OF THE PREFERRED EMBODIMENT

The invention is further explained below in conjunction with the drawings and the embodiments.

In the present invention, the distal end refers to the end away from the surgical operator, and the proximal end refers to the end close to the surgical operator.

### Embodiment 1

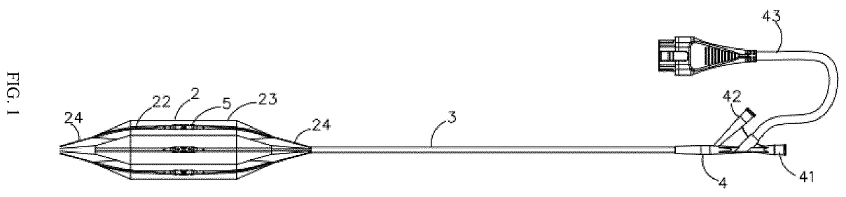

As shown in FIG. 1 to FIG. 3, the embodiment 1 discloses a shock wave balloon dilation catheter, which sequentially comprises, from the distal end to the proximal end, a tip (1), a balloon (2), a dilation catheter (3), and a handle base (4), wherein:
The balloon (2) is composed of three annularly distributed balloon bodies (23), the three balloons bodies (23) are closely attached to each other, and recesses are formed between the outer circumferences of the two adjacent balloon bodies (23), forming a blood flow passage (21). The blood flow passage (21) allows a certain level of cardiac output to be maintained during balloon dilation while treating heart valve calcification, avoiding complete blockage of blood flow channel, thereby reducing the impact of the balloon dilation procedure on hemodynamics and lowering the incidence of circulatory interruption. Both the proximal end and the distal end of the balloon bodies (23) are provided with support rods (24). The support rods (24) at the proximal end are formed by branches from the outer wall of the dilation catheter (3), and the support rods (24) at the proximal end are provided with a support rod cavity (241). The chamber of the balloon bodies (23) is provided with a fluid-conducting tube (22). The fluid-conducting tube (22) has a certain elasticity. The distal end of the fluid-conducting tube (22) is a sealed surface, which is fixedly connected to the distal end of the chamber of the balloon body (23). The proximal end of the fluid-conducting tube (22) is connected and sealed to the support rod cavity (241) so that the conductive fluid does not directly enter the balloon body (23) from the support rod cavity (241). The fluid-conducting tube (22) is provided with through-holes (221), allowing conductive fluid to enter the balloon bodies (23) from the support rod cavities (241) through the fluid-conducting tubes (22) via the through-holes (221), thereby enabling the balloon bodies (23) to inflate or deflate. Shock wave generator (5) is provided on the fluid-conducting tube (22), the emission direction of the shock wave generator (5) is facing the outer circumference of the balloon (2);
The distal end of the dilation catheter (3) passes through the space between the three balloon bodies (23) and is fixedly connected to the distal tip (1). The dilation catheter (3) is provided with a guide wire lumen (31) and a fluid passage (32). The proximal end of the dilation catheter (3) is connected to the handle base (4), and the distal support rod (24) is inserted between the dilation catheter (3) and the tip (1), and is fixed to each other. The proximal end of the proximal support rod (24) is fixedly connected to the outer wall of the dilation catheter (3), and the fluid passage (32) penetrates through the outer wall of the dilation catheter (3) and communicates with the support rod cavity (241);
The tip (1) has a cavity, and the guide wire lumen (31) communicates with the cavity of the tip (1). The tip (1) is composed of a conical section and a cylindrical section, where the cylindrical section is fixedly connected to the distal end of the dilation catheter (3), and the conical section is arranged at the distal end of the cylindrical section;
The handle base (4) comprises a first interface (41) and a second interface (42), and the handle base (4) is provided with two channels respectively connecting the first interface (41) and the second interface (42). The first interface (41) communicates with the guide wire lumen (31) through one of the channels, while the second interface (42) communicates with the fluid passage (32) through the other channel. The handle base (4) further comprises an electrical connector (43), and the electrical connector (43) is electrically connected to the shock wave generator (5) through conducting wires (6), allowing simultaneous operation of the shock wave generator (5).

By adopting the above structure, there is no need to separately set up a blood flow passage; the blood flow passage is naturally formed by the shape of the balloon. This design meets the requirements for pre-treatment of the tricuspid valve opening while also resolving the issue of vascular blockage.

As shown in FIG. 3, in the embodiment 1, the balloon (2) is generally oval-shaped.

As shown in FIG. 4 to FIG. 5, the shock wave generator (5) comprises an electrode ring (51), an insulating sleeve (52), and a metal sleeve (53). The electrode ring (51) is fitted over the fluid-conducting tube (22), and the insulating sleeve (52) is arranged in between the electrode ring (51) and the fluid-conducting tube (22). The conducting wires (6) are coated with an insulating layer, and exposed copper sections are provided on the conducting wires (6). The metal sleeve (53) is fitted over the exposed copper sections and is fixed tightly in place. The electrode ring (51) is provided with at least one electrode ring discharge through-hole (511), and the electrode ring discharge through-holes (511) are facing the outer circumference of the balloon (2). The exposed copper sections of the conducting wires are aligned with the electrode ring discharge through-holes (511). Exposed holes (521) that expose part of the metal sleeve (53) are provided at the location where the insulating sleeve (52) is located at the electrode ring discharge through-holes (511). The gaps between the electrode ring (51), the insulating sleeve (52), and the metal sleeve (53) are filled with the insulating glue (54) and fixedly attached to the outer surface of the fluid-conducting tube (22), thereby strengthening the structure and ensuring uniform shock wave emission.

As shown in FIG. 3, the shock wave generator (5) is arranged at the center of the balloon body (23).

As shown in FIG. 4 to FIG. 5, each of the shock wave generators (5) consists of two symmetrically arranged ring bodies (512), a connecting portion (513) is provided between the two ring bodies (512), and the two ring bodies (512) are connected by the connecting portion (513). The electrode ring discharge through-holes (511) are respectively provided on the two ring bodies (512), allowing each of the shock wave generators (5) to release two pulses of shock wave energy, with two electrode ring discharge through-holes (511) positioned at the same location.

In the embodiment 1, the shock wave generators (5) are connected in series, and the conducting wires (6) comprise a positive wire (61), a negative wire (62), and a connecting wire (63). The positive wire (61) is aligned with the proximal ring body (512) of one of the shock wave generator (5), while the negative wire (62) is aligned with the proximal ring body (512) of the another shock wave generator (5). The connecting wire (63) connects the two shock wave generators (5) in series. Specifically, for clarity, the shock wave generators (5) in each of the balloon bodies are respectively defined as the first shock wave generator (5A), the second shock wave generator (5B), and the third shock wave generator (5C). The distal end of the positive wire (61) has an exposed copper section aligned with the electrode ring discharge through-hole (511) on the proximal ring body (512) of the first shock wave generator (5A). Both ends of the connecting wire (63) have exposed copper sections. There are two connecting wires (63), one end of one of the connecting wires is aligned with the electrode ring discharge through-hole (511) on the distal ring body (512) of the first shock wave generator (5A), while the other end is aligned with the electrode ring discharge through-hole (511) on the distal ring body (512) of the second shock wave generator (5B). One end of the other connecting wires is aligned with the electrode ring discharge through-hole (511) on the distal ring body (512) of the second shock wave generator (5B), while the other end is aligned with the electrode ring discharge through-hole (511) on the proximal ring body (512) of the third shock wave generator (5C). The distal end of the negative wire (62) has an exposed copper section aligned with the electrode ring discharge through-hole (511) on the distal ring body (512) of the third shock wave generator (5C). The proximal end of the positive wire (61) and the proximal end of the negative wire (62) are electrically connected to the positive and negative terminals of the electrical connector (43), respectively.

The positive wire (61) and negative wire (62) respectively enter the balloon body (23) through the support rod cavity (241) of the proximal support rod (24). The connecting wires (63) can be arranged by attaching to the outer wall of the dilation catheter (3) and is fixed in place using glue.

As shown in FIG. 4 to FIG. 5, each of the fluid-conducting tubes (22) is provided with two through-holes (221).

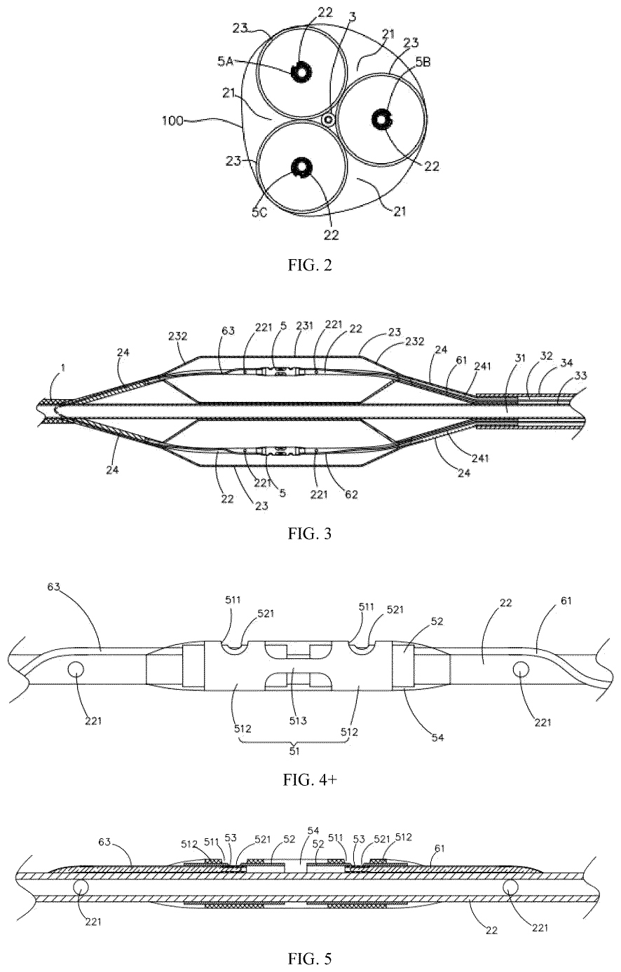

As shown in FIG. 2, the balloon body (23) comprises a cylindrical section (231) located in the middle, and conical sections (232) located at the proximal end and the distal end of the cylindrical section (231), respectively.

As shown in FIG. 2, the dilation catheter (3) comprises an inner tube (33) and an outer tube (34). The outer tube (34) has a larger diameter than the outer diameter of the inner tube (33). The outer tube (34) is fitted over the inner tube (33), and the outer tube (34) is coaxially arranged with the inner tube (33). The fluid passage (32) is arranged between the inner tube (33) and the outer tube (34), while the guide wire lumen (31) is formed by the cavity of the inner tube (33). The distal end of the inner tube (33) extends beyond the distal end of the outer tube (34), and fixedly connects to the tip (1) after passing through the space between the three balloon bodies (23). The distal end of the fluid passage (32) is a sealed surface. The proximal end of the support rod (24) is connected to the distal end of the fluid passage (32) and a through-hole is provided at the distal end of the fluid passage (32) to connect the support rod lumen (241) with the fluid passage (32), allowing communication between the proximal end of the support rod (24) and the fluid passage (32). The proximal end of the inner tube (33) is sealedly connected to the channel of the first interface (41), while the proximal end of the outer tube (34) is sealedly connected to the channel of the second interface (42).

The positive wire (61) and negative wire (62) can be extended through the fluid passage (32) to the handle base (4) and electrically connected to the electrical connector (43). Alternatively, separate wire channels can be arranged within the fluid passage (32) and manufactured using co-extrusion.

In the embodiment 1, the electrode ring (51) is made from a conductive metal material, preferably 304 stainless steel. The insulating sleeve (52) is made from an insulating material, such as PI (polyimide). The metal sleeve (53) is also made from a conductive metal material, preferably 304 stainless steel. The insulating glue (54) is an insulating UV adhesive, such as Loctite 349 UV glue.

During use, the embodiment 1 is positioned at the tricuspid valve. After introducing a conductive fluid, the balloon body is inflated. Once the tricuspid valve is expanded, the space between the tricuspid valve wall (100) and the recessed portion (the blood flow passage (21)) is not completely blocked (as shown in FIG. 2), allowing blood flow at the blood flow passage (21) formed by the recessed portion. Upon triggering the shock wave generator (5), shock wave energy is released around, thereby shattering the calcified portions and restoring the physiological function of the tricuspid valve. This approach allows for the repair of the tricuspid valve without the need for an artificial valve, and ensures that blood flow is not completely blocked during the repair process, enhancing the safety of the procedure.

### Embodiment 2

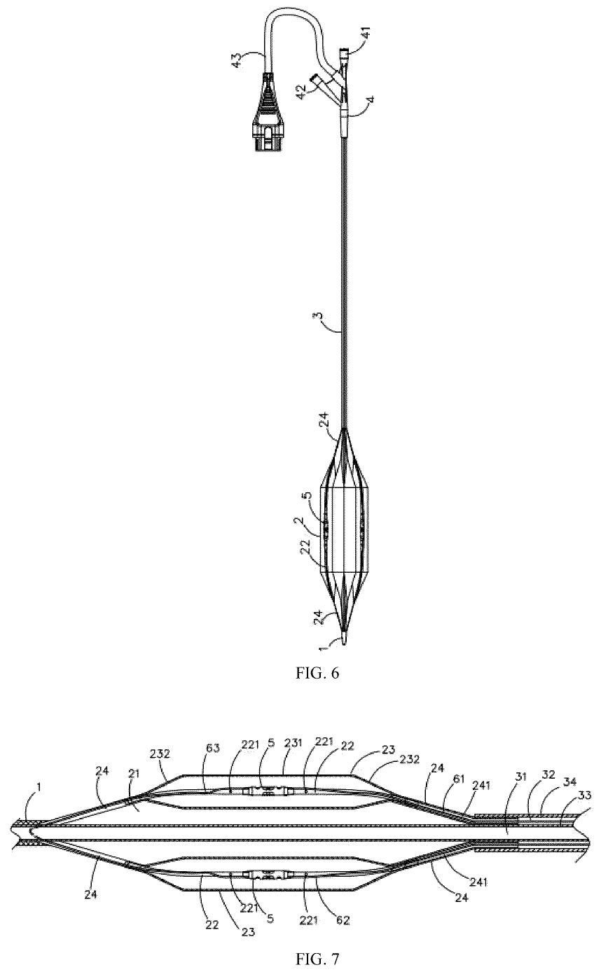

As shown in FIG. 6 to FIG. 8, the embodiment 2 discloses a shock wave balloon dilation catheter, which sequentially comprises, from the distal end to the proximal end, a tip (1), a balloon (2), a dilation catheter (3), and a handle base (4), wherein:
The balloon (2) consists of eight balloon bodies (23), and the balloon bodies are distributed annularly around the dilation catheter (3), forming a ring shape. A blood flow passage (21) is provided at the center of the balloon (2), the blood flow passage (21) is arranged axially along the balloon (2), and penetrates through the balloon (2). The blood flow passage (21) allows a certain level of cardiac output to be maintained during balloon dilation while treating heart valve calcification, avoiding complete blockage of blood flow channel, thereby reducing the impact of the balloon dilation procedure on hemodynamics and lowering the incidence of circulatory interruption. The balloon (2) is generally oval-shaped, and the proximal end and the distal end of the balloon body (23) are provided with support rods (24), respectively. The distal end of the balloon body (23) is a sealed surface. The support rods (24) are fixedly connected to the tip (1) and the dilation catheter (3), respectively. A shock wave generator (5) is provided in the chambers of at least two of the balloon bodies (23), and the balloon bodies (23) with the shock wave generators (5) are provided with fluid-conducting tubes (22). The fluid-conducting tubes (22) are provided with through-holes (221), and the distal end of the fluid-conducting tube (22) is a sealed end, and is fixedly connected to the distal end of the chamber of the balloon (2), while the proximal end of the fluid-conducting tube (22) is fixed to the proximal end of the chamber of the balloon body (23). Each of the support rods (24) at the proximal end is provided with a support rod cavity (241), and the proximal end of the support rod cavity (241) communicates with the fluid passage (32) in the dilation catheter (3). The proximal end of the fluid-conducting tube (22) in the balloon body (23) with the shock wave generators (5) communicates with the support rod cavity (241), while the proximal end of the chamber of the balloon body (23) with shock wave generators (5) is sealed with the proximal end of the fluid-conducting tube (22), allowing the balloon bodies without the shock wave generators (5) to inflate by introducing conductive fluid or deflate by withdrawing conductive fluid through the support rod cavity (241). The balloon bodies with the shock wave generators (5) inflate by introducing conductive fluid through the through-holes (221) in the fluid-conducting tube (22) or deflate by withdrawing conductive fluid. The chamber of the balloon (2) is provided with a flexible fluid-conducting tube (22) that can axially stretch when the fluid-conducting tube (22) dilates in the balloon (2), and the shock wave generators (5) are fixed in the chamber of the balloon (2) by the fluid-conducting tubes (22);
The distal end of the dilation catheter (3) passes through the blood flow passage (21) and is fixedly connected to the proximal end of the tip (1) and the closed end of the balloon (2). The outer diameter of the dilation catheter (3) is smaller than the diameter of the blood flow passage (21). The proximal ends of the balloon (2) and fluid-conducting tubes (22) are fixed to the outer wall of the dilation catheter (3). The proximal end of the dilation catheter (3) is fixedly connected to the distal end of the handle base (4), and the dilation catheter (3) is further provided with a guide wire lumen (31);
The tip (1) has a cavity, and the guide wire lumen (31) communicates with the cavity of the tip (1). The tip (1) is composed of a conical section and a cylindrical section, where the cylindrical section is fixedly connected to the distal end of the dilation catheter (3), and the conical section is arranged at the distal end of the cylindrical section;
The handle base (4) comprises a first interface (41) and a second interface (42), and the handle base (4) is provided with two channels respectively connecting the first interface (41) and the second interface (42). The first interface (41) communicates with the guide wire lumen (31) through one of the channels, while the second interface (42) communicates with the fluid passage (32) through the other channel. The handle base (4) further comprises an electrical connector (43), the conducting wires (6) of the shock wave generator (5) extend along the outer wall of the fluid-conducting tube (22) through the fluid passage (32) to the handle base (4) and are electrically connected to the electrical connector (43). The conducting wires can be extended along the outer walls of both the fluid-conducting tube (22) and dilation catheter (3), or manufactured using co-extrusion.

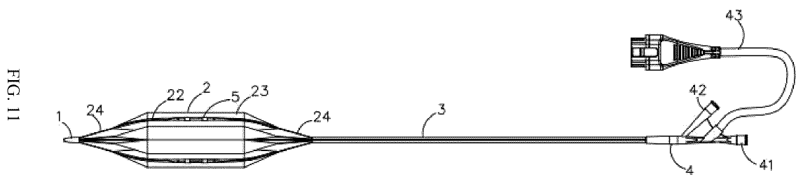

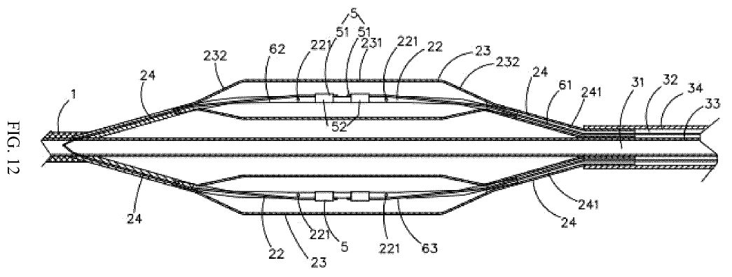

As shown in FIG. 9 to FIG. 10, the shock wave generator (5) comprises an electrode ring (51), an insulating sleeve (52), and a metal sleeve (53). The electrode ring (51) is fitted over the fluid-conducting tube (22), and the insulating sleeve (52) is arranged in between the electrode ring (51) and the fluid-conducting tube (22). The conducting wires (6) are coated with an insulating layer, and exposed copper sections are provided on the conducting wires (6). The metal sleeve (53) is fitted over the exposed copper sections and is fixed tightly in place. The electrode ring (51) is provided with at least one electrode ring discharge through-hole (511), and the electrode ring discharge through-holes (511) are facing the outer circumference of the balloon (2). The exposed copper sections of the conducting wires are aligned with the electrode ring discharge through-holes (511). Exposed holes (521) that expose part of the metal sleeve (53) are provided at the location where the insulating sleeve (52) is placed with the electrode ring discharge through-holes (511). The gaps between the electrode ring (51), the insulating sleeve (52), and the metal sleeve (53) are filled with the insulating glue (54) and fixedly attached to the outer surface of the fluid-conducting tube (22), thereby the strengthen the structure and ensuring uniform shock wave emission.

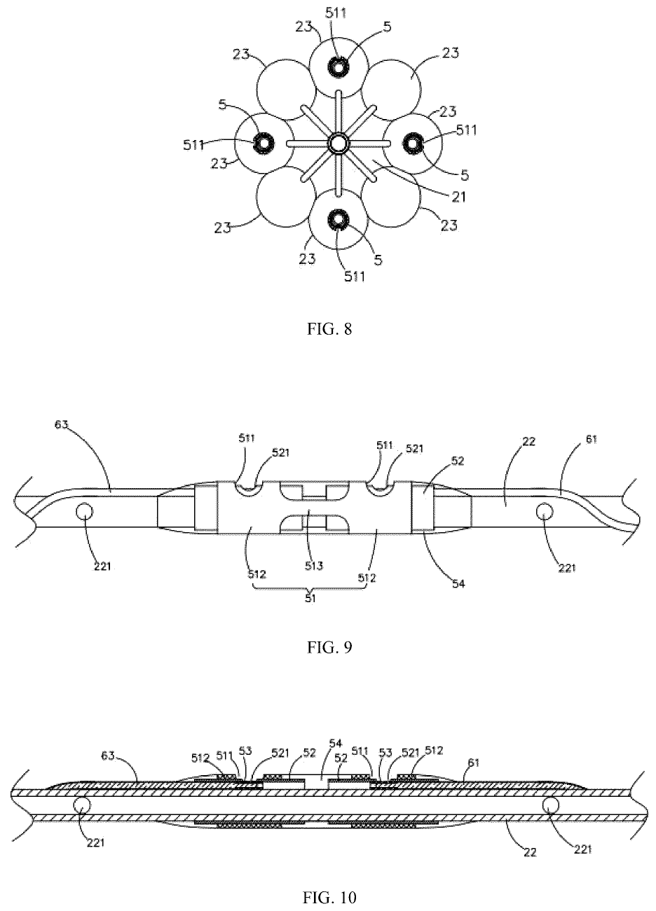

As shown in FIG. 8, the shock wave generator (5) is arranged at the center of the balloon body (23).

As shown in FIG. 9 to FIG. 10, each of the shock wave generators (5) consists of two symmetrically arranged ring bodies (512), a connecting portion (513) is provided between the two ring bodies (512), and the two ring bodies (512) are connected by the connecting portion (513). The electrode ring discharge through-holes (511) are respectively provided on the two ring bodies (512), allowing each of the shock wave generators (5) to release two pulses of shock wave energy, with two electrode ring discharge through-holes (511) positioned at the same location.

In the embodiment 2, at least one balloon body (23) without the shock wave generator (5) is arranged between the adjacent balloon bodies (23) that have the shock wave generators (5). The shock wave generators (5) are arranged in pairs aligned with each other, and the two aligned shock wave generators (5) are connected in series. The conducting wires (6) comprise a positive wire (61), a negative wire (62), and a connecting wire (63). The positive wire (61) is aligned with the proximal ring body (512) of one of the shock wave generator (5), while the negative wire (62) is aligned with the proximal ring body (512) of the another shock wave generator (5). The connecting wire (63) is arranged between the distal ring bodies (512) of the two shock wave generators (5). Wherein, the distal end of the positive wire (61) has an exposed copper section aligned with the electrode ring discharge through-hole (511) of one of the shock wave generators (5), while both ends of the connecting wire (63) have exposed copper sections aligned with the electrode ring discharge through-holes (511) of the distal ring bodies (512) of both of the shock wave generators (5). The distal end of the negative wire (62) has an exposed copper section aligned with the electrode ring discharge through-hole (511) of the proximal ring body (512) of the other shock wave generator (5).

The positive wire (61) and negative wire (62) respectively enter the balloon body (23) through the support rod cavity (241) of the proximal support rod (24). The connecting wires (63) is attached to the outer wall of the dilation catheter (3) and is arranged at the tip (1), and is fixed in place using glue.

As shown in FIG. 9 to FIG. 10, each of the fluid-conducting tubes (22) is provided with two through-holes (221).

As shown in FIG. 8, the number of the balloon bodies (23) can also be an even number of four or more, such as 4, 6, 8, etc., and can be understood as a multiple of 2.

In the embodiment 2, the number of the shock wave generators (5) is set to an even number of more than two, such as 2, 4, or 6, and the number of the shock wave generators (5) is smaller than the number of the balloon bodies (23).

In one embodiment of the embodiment 2, as shown in FIG. 8, four shock wave generators (5) are provided, and are arranged in pairs aligned with each other, respectively. As depicted, there is one balloon body (23) without a shock wave generator (5) placed between each pair of adjacent shock wave generators (5).

As shown in FIG. 7, the balloon body (23) comprises a cylindrical section (231) located in the middle, and conical sections (232) located at the proximal end and the distal end of the cylindrical section (231), respectively.

As shown in FIG. 7, the dilation catheter (3) comprises an inner tube (33) and an outer tube (34). The outer tube (34) has a larger diameter than the outer diameter of the inner tube (33). The outer tube (34) is fitted over the inner tube (33), and the outer tube (34) is coaxially arranged with the inner tube (33). The fluid passage (32) is arranged between the inner tube (33) and the outer tube (34), while the guide wire lumen (31) is formed by the cavity of the inner tube (33). The distal end of the inner tube (33) extends beyond the distal end of the outer tube (34), and fixedly connects to the tip (1) after passing through the blood flow passage (21). The proximal end of the support rod (24) is inserted into and fixed within the fluid passage (32) from the distal end of the outer tube (34), ensuring that the proximal end of the support rod (24) communicates with the fluid passage (32). The distal port of the fluid passage (32) is sealedly arranged.

The positive wire (61) and negative wire (62) can be extended through the fluid passage (32) to the handle base (4) and electrically connected to the electrical connector (43). Alternatively, separate wire channels can be arranged within the fluid passage (32) and manufactured using co-extrusion.

In the embodiment 2, the electrode ring (51) is made from a conductive metal material, preferably 304 stainless steel. The insulating sleeve (52) is made from an insulating material, such as PI (polyimide). The metal sleeve (53) is also made from a conductive metal material, preferably 304 stainless steel. The insulating glue (54) is an insulating UV adhesive, such as Loctite 349 UV glue.

During use, the embodiment 2 is positioned at the tricuspid valve. After introducing a conductive fluid, the balloon body is inflated. Once the tricuspid valve is expanded, the shock wave generator (5) is triggered, shock wave energy is released around, thereby shattering the calcified portions and restoring the physiological function of the tricuspid valve. This approach allows for the repair of the tricuspid valve without the need for an artificial valve, enhancing the safety of the procedure.

### Embodiment 3

As shown in FIG. 11 to FIG. 13, the embodiment 3 discloses a shock wave balloon dilation catheter, which sequentially comprises, from the distal end to the proximal end, a tip (1), a balloon (2), a dilation catheter (3), and a handle base (4), wherein:
The balloon (2) consists of eight balloon bodies (23), and the balloon bodies are distributed annularly around the dilation catheter (3), forming a ring shape, thereby forming a blood flow passage (21) at the center of the balloon (2). The balloon (2) is generally oval-shaped, and the blood flow passage (21) is arranged axially along the balloon (2). The blood flow passage (21) allows a certain level of cardiac output to be maintained during balloon dilation while treating heart valve calcification, avoiding complete blockage of blood flow channel, thereby reducing the impact of the balloon dilation procedure on hemodynamics and lowering the incidence of circulatory interruption. Both the proximal end and the distal end of the balloon bodies (23) are provided with support rods (24), respectively. The support rods (24) are fixedly connected to the tip (1) and dilation catheter (3), respectively. Shock wave generators (5) are provided in the chambers of at least two of the balloon bodies (23) that are aligned with each other, and the balloon bodies (23) with the shock wave generators (5) are provided with fluid-conducting tubes (22). The distal end of the fluid-conducting tube (22) is fixed to the distal end of the chamber of the balloon body (23), while the proximal end of the fluid-conducting tube (22) is fixed to the proximal end of the chamber of the balloon body (23). The fluid-conducting tubes (22) are provided with through-holes (221), and the support rod (24) at the proximal end is provided with a support rod cavity (241), and communicates with the chamber of the balloon body (23). The proximal end of the support rod cavity (241) communicates with the distal end of the fluid passage (32). Wherein, in the balloon bodies (23) without the shock wave generators (5), the proximal end of the support rod cavity (241) directly communicates with the distal end of the fluid passage (32). In balloon bodies (23) with shock wave generators (5), the proximal end of the fluid-conducting tube (22) is in communication with and seals to the distal end of the support rod cavity (241), and the outer wall of the proximal end of the fluid-conducting tube (22) is sealed to the proximal end of the chamber of the balloon body (23), allowing the balloon bodies without the shock wave generators (5) to inflate by introducing conductive fluid or deflate by withdrawing conductive fluid through the support rod cavity (241). The balloon bodies with the shock wave generators (5) inflate by introducing conductive fluid through the through-holes (221) in the fluid-conducting tube (22) or deflate by withdrawing conductive fluid. Arranging eight balloon bodies (23) provides better expansion of the tricuspid valve;
The distal end of the dilation catheter (3) passes through the blood flow passage (21) and is fixedly connected to the proximal end of the tip (1). The distal support rod (24) is inserted between the tip (1) and the dilation catheter (3) and is fixed in place. The proximal end of the dilation catheter (3) is fixedly connected to the distal end of the handle base (4). The dilation catheter (3) is provided with a guide wire lumen (31) and a fluid passage (32), and the fluid passage (32) communicates with the support rod cavity (241) of the support rod (24) at the proximal end;
The tip (1) has a cavity, and the guide wire lumen (31) communicates with the cavity of the tip (1). The tip (1) is composed of a conical section and a cylindrical section, where the cylindrical section is fixedly connected to the distal end of the dilation catheter (3), and the conical section is arranged at the distal end of the cylindrical section;
The handle base (4) comprises a first interface (41) and a second interface (42), and the handle base (4) is provided with two channels respectively connecting the first interface (41) and the second interface (42). The first interface (41) communicates with the guide wire lumen (31) through one of the channels, while the second interface (42) communicates with the fluid passage (32) through the other channel. The handle base (4) further comprises an electrical connector (43), the conducting wires (6) of the shock wave generator (5) extend along the outer wall of the fluid-conducting tube (22) through the fluid passage (32) to the handle base (4) and are electrically connected to the electrical connector (43).

In the embodiment 3, the balloon body (23) and the support rod are integrated as a single structure.

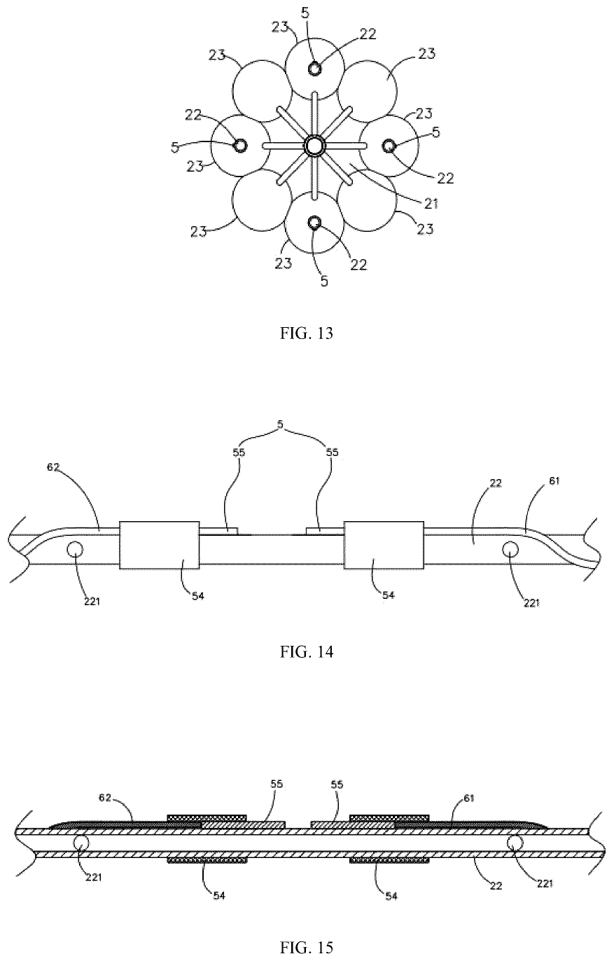

As shown in FIG. 13, the shock wave generator (5) is arranged at the center of the balloon body (23).

As shown in FIG. 14 to FIG. 15, each of the shock wave generators (5) comprises at least one pair of electrode plates (55), the electrode plates (55) are fixed on the side of the fluid-conducting tube (22) facing the outer circumference of the balloon (2), enabling the shock waves to be emitted towards the outer circumference of the balloon (2). A gap is arranged between the pair of electrode plates (55), and the shock wave generator (5) is electrically connected to the conducting wires (6). The use of electrode plates is advantageous as the structure of the electrode plates is simple and low implementation difficulty. The plasma arc generated between the opposing electrode plates in the axial direction produces a pulse waveform that is conducive to the propagation of transverse waves while minimizing interference from longitudinal waves, facilitating the shattering of calcification.

In the embodiment 3, the electrode plates (55) are sheet-shaped, and are fixedly attached to the outer wall of the fluid-conducting tube (22) using the insulating glue (54), the insulating glue (54) partially wraps the electrode plates (55), leaving only the ends of the electrode plates (55) that are aligned with each other exposed.

In the embodiment 3, the shock wave generators (5) can be electrically connected to the electrical connector (43) in series or in parallel.

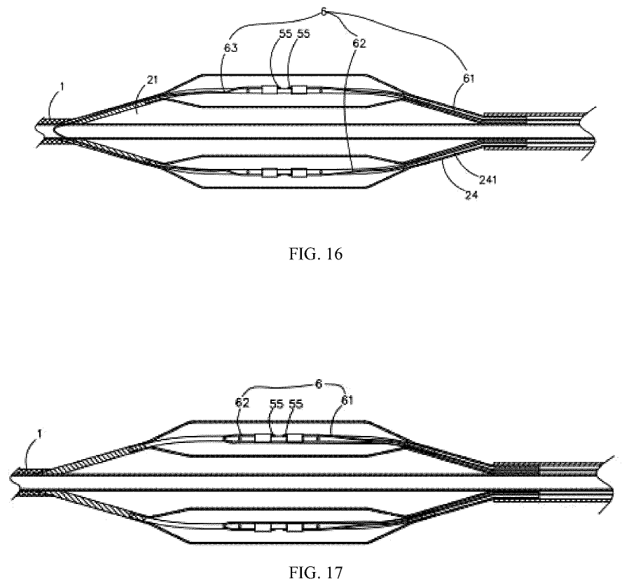

When in series connection, as shown in FIG. 16, the conducting wires (6) comprise a positive wire (61), a connecting wire (63) and a negative wire (62), one of the electrode plate (55) from a pair of electrode plates (55) is electrically connected to the positive wire (61), while the other electrode plate (55) in the same pair is electrically connected to one electrode plate (55) in another pair of electrode plates (55) through the connecting wire (63), and the remaining electrode plate (55) in that second pair of electrode plates (55) is electrically connected to the negative wire (62), forming a series connection.

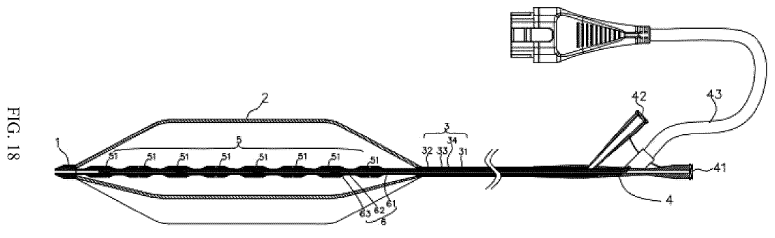

When in parallel connection, as shown in FIG. 17, at least two sets of conducting wires (6) are provided, with each set comprising a positive wire (61) and a negative wire (62), one pair of electrode plates (55) is electrically connected to one set of the positive wire (61) and the negative wire (62), respectively, while another pair of the electrode plates (55) is electrically connected to another set of the positive wire (61) and the negative wire (62), respectively, forming a parallel connection.

In the embodiment 3, the positive wire (61) and the negative wire (62) respectively pass through the support rod cavities (241) of the respective proximal support rods (24) and enter the balloon bodies (23). The connecting wires (63) is attached to the outer wall of the dilation catheter (3) and passed through the tip (1), with the connecting wires (63) being fixed in place using glue.

All wires (6) are provided with an insulating outer layer.

In the embodiment 3, the number of the shock wave generators (5) is set to an even number of more than two, such as 2, 4, or 6, and the number of the shock wave generators (5) is smaller than the number of the balloon bodies (23).

As shown in FIG. 12, the balloon body (23) comprises a cylindrical section (231) located in the middle, and conical sections (232) located at the proximal end and the distal end of the cylindrical section (231), respectively.

As shown in FIG. 12, the dilation catheter (3) comprises an inner tube (33) and an outer tube (34). The outer tube (34) has a larger diameter than the outer diameter of the inner tube (33). The outer tube (34) is fitted over the inner tube (33), and the outer tube (34) is coaxially arranged with the inner tube (33). The fluid passage (32) is arranged between the inner tube (33) and the outer tube (34), while the guide wire lumen (31) is formed by the cavity of the inner tube (33). The distal end of the inner tube (33) extends beyond the distal end of the outer tube (34), and is fixedly connected to the tip (1) after passing through the blood flow passage (21). The distal end of the fluid passage (32) is a sealed surface. The proximal end of the support rod (24) is connected to the distal end of the fluid passage (32), and a through-hole is provided at the distal end of the fluid passage (32) to connect the support rod lumen (241) with the fluid passage (32).

The positive wire (61) and negative wire (62) can be extended through the fluid passage (32) to the handle base (4) and electrically connected to the electrical connector (43). Alternatively, separate wire channels can be arranged within the fluid passage (32) and manufactured using co-extrusion.

In the embodiment 3, the electrode plate (55) is made from a conductive metal material, preferably 304 stainless steel. The insulating glue (54) is an insulating UV adhesive, such as Loctite 349 UV glue.

During use, the embodiment 3 is positioned at the tricuspid valve. After introducing a conductive fluid, the balloon body is inflated. Once the tricuspid valve is expanded, the shock wave generator (5) is triggered, shock wave energy is released around, thereby shattering the calcified portions and restoring the physiological function of the tricuspid valve. This approach allows for the repair of the tricuspid valve without the need for an artificial valve, enhancing the safety of the procedure.

### Embodiment 4

As shown in FIG. 18 to FIG. 20, the embodiment 4 discloses a shock wave balloon dilation catheter, which sequentially comprises, from the distal end to the proximal end, a tip (1), a balloon (2), a dilation catheter (3), and a handle base (4), wherein:

The outer wall of the balloon (2) is provided with at least three arcuate protrusions (25) distributed circumferentially along the balloon (2). The inner cavities of the arcuate protrusions (25) communicate with the inner cavity of the balloon (2), forming an integral structure. arcuate recesses are provided between each pair of adjacent arcuate protrusions (25), forming a blood flow passage (21), and the arcuate protrusions (25) extend axially along the balloon (2);
The dilation catheter (3) is provided with a guide wire lumen (31) and a fluid passage (32). The guide wire lumen (31) is arranged along the axis of the dilation catheter (3), while the fluid passage (32) surrounds the outer circumference of the guide wire lumen (31). The proximal end of the dilation catheter (3) is connected to the handle base (4), while the distal end of the dilation catheter (3) passes through the inner cavity of the balloon (2), extending from the distal end of the balloon (2) and connecting to the tip (1). The dilation catheter (3) is coaxially arranged with the balloon (2). The proximal end of the balloon (2) is connected to the outer wall of the dilation catheter (3), while the distal end of the balloon (2) is connected to the tip (1). The fluid passage (32) communicates with the inner cavity of the balloon (2), allowing the balloon (2) to be inflated through the fluid passage (32). When the balloon (2) is inflated, there remains a certain gap between the arcuate recess (i.e., the blood flow passage (21)) and the tricuspid valve opening, allowing blood flow to pass through the blood flow passage (21);
The tip (1) has a cavity, and the guide wire lumen (31) communicates with the cavity of the tip (1). The tip (1) is composed of a conical section and a cylindrical section, where the cylindrical section is fixedly connected to the distal end of the dilation catheter (3), and the conical section is arranged at the distal end of the cylindrical section;
A plurality of sets of shock wave generators (5) with different emission directions are arranged on the section of the dilation catheter (3) located within the balloon (2), enabling shock wave energy to be emitted in all directions toward the outer circumference of the balloon (2);
The handle base (4) comprises a first interface (41) and a second interface (42), and the handle base (4) is provided with two channels respectively connecting the first interface (41) and the second interface (42). The first interface (41) communicates with the guide wire lumen (31) through one of the channels, while the second interface (42) communicates with the fluid passage (32) through the other channel. The handle base (4) further comprises an electrical connector (43), and the electrical connector (43) is electrically connected to the shock wave generator (5) through conducting wires (6).

By adopting the above structure, there is no need to separately set up a blood flow passage; the blood flow passage (21) is naturally formed by the shape of the balloon. Blood flow passes through the arcuate recesses of the balloon, while the arcuate protrusions (25) is used to secure the balloon to the tricuspid valve opening wall.

As shown in FIG. 18, the balloon (2) of the present invention is generally oval-shape.

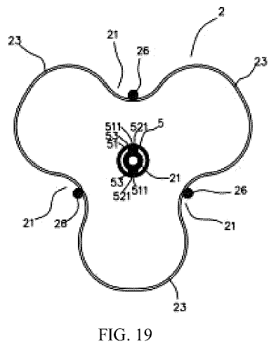

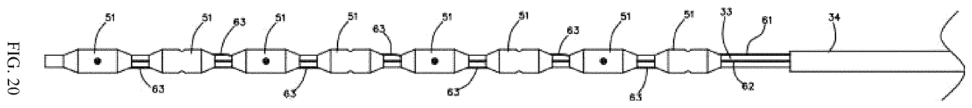

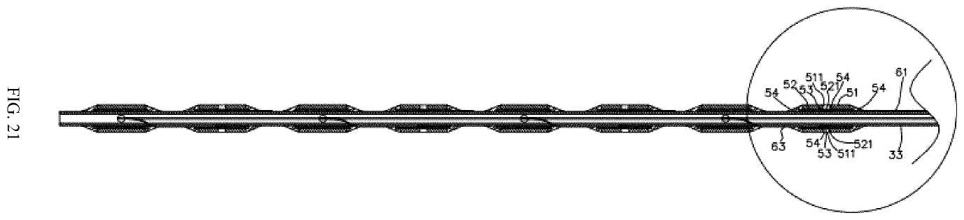

As shown in FIG. 19, reinforcing ribs (26) are provided on the arcuate recesses, the reinforcing ribs (26) are arranged axially along the balloon (2). The reinforcing ribs (26) can be fixedly attached using glue, or woven into the body of the balloon (2) during the balloon weaving process. Preferably, the reinforcing ribs (26) are arranged at the center of the arcuate recesses. In the embodiment 4, each of the arcuate recesses is provided with one reinforcing rib (26). During the inflation process of conventional balloons, the outer wall of the balloons will expand outward equivalently. By incorporating reinforcing ribs, the tendency for the balloon wall to expand at the rib positions can be limited, allowing the protrusions and recesses of the balloon to be achieved.

As shown in FIG. 19, the arcuate protrusions (25) are preferably arc-shaped. The shapes of the three arcuate protrusions (25) are identical. The arcuate recesses transition uniformly and integrate into a single unit with the adjacent arcuate protrusions (25).

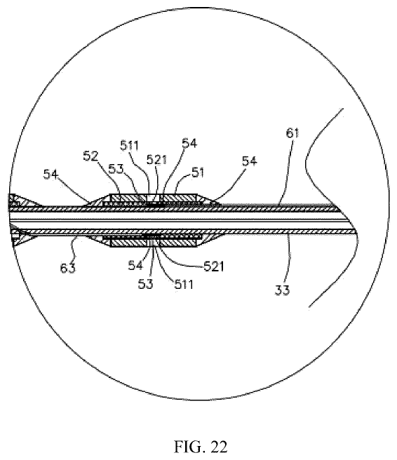

As shown in FIG. 20 to FIG. 22, the shock wave generator (5) comprises a plurality of electrode rings (51), insulating sleeves (52), and metal sleeves (53). The number of the insulating sleeves (52) is the same as the number of the electrode rings (51). The conducting wires (6) is provided with exposed copper sections. The metal sleeves (53) are fitted over the exposed copper sections of the conducting wires (6) and are fixed in place with the insulating glue (54) on the outer wall of the dilation catheter (3). The insulating sleeves (52) are fitted over the portion of the dilation catheter (3) outside the metal sleeves (53) to cover the metal sleeves (53). The electrode rings (51) are fitted over the insulating sleeves (52) and are fixed in place with the insulating glue (54). Insulating sleeve through-holes (521) that exposes the metal sleeves (53) are provided at the location where the insulating sleeves (52) are located at the metal sleeves (53), and electrode ring discharge through-holes (511) are provided at the location where the insulating sleeve through-hole (521) aligned with the electrode rings (51). The electrode ring discharge through-holes (511) are provided with two, symmetrically arranged on the electrode ring (51). Correspondingly, the insulating sleeve through-holes (521) are also provided with two, arranged at the location that aligned with the electrode ring discharge through-holes (511).

The conducting wires (6) comprise a positive wire (61), a negative wire (62), and a connecting wire (63). The connecting wire (63) is arranged between adjacent electrode rings (51), and the exposed copper sections of the connecting wire (63) are aligned with one of the electrode ring discharge through-holes (511). Each of the exposed copper sections is covered with the metal sleeve (53). The distal end of the positive wire (61) extends along the dilation catheter (3) to the electrode ring (51) of the nearest end. The distal end of the positive wire (61) is provided with a copper exposed section, and the copper exposed section is also covered with the metal sleeve (53). The copper exposed section of the positive wire (61) is aligned with one of the electrode ring discharge through-holes (511) on the nearest electrode ring (51). The proximal end of the positive wire (61) extends to the positive electrode of the electrical connector (43) and is electrically connected. The distal end of the negative wire (62) is also provided with a copper exposed section, and the copper exposed section is provided with a metal sleeve (53). The distal end of the negative wire (62) is aligned with one of the electrode ring discharge through-holes (511) of the electrode ring (51) on the farthest end. The proximal end of the negative wire (62) extends along the dilation catheter (3) to the negative electrode of the electrical connector (43) and is electrically connected.

Taking one of the electrode rings (51) as an example, when the proximal end of the connecting wire (63) on the distal end of the electrode ring (51) is aligned with one of the electrode ring discharge through-holes (511) on that electrode ring (51), the distal end of the connecting wire (63) on the proximal end of the electrode ring (51) will be aligned with another electrode ring discharge through-hole (511). In other words, the two exposed copper sections on the connecting wire (63) correspond to one of the electrode ring discharge through-holes (511) of two electrode rings (51), respectively.

In the embodiment 4, the adjacent electrode ring discharge through-holes (511) on the electrode rings (51) are arranged in a staggered manner.

As shown in FIG. 19, the shock wave generator (5) is arranged at the center of the balloon body (23).

As shown in FIG. 18, the dilation catheter (3) comprises an inner tube (33) and an outer tube (34). The outer tube (34) has a larger diameter than the outer diameter of the inner tube (33). The outer tube (34) is fitted over the inner tube (33), and the outer tube (34) is coaxially arranged with the inner tube (33). The fluid passage (32) is arranged between the inner tube (33) and the outer tube (34), while the guide wire lumen (31) is formed by the cavity of the inner tube (33). The distal end of the inner tube (33) extends beyond the distal end of the outer tube (34), and fixedly connects to the tip (1) after passing through the blood flow passage (21). The distal end of the outer tube (34) connects with the proximal end of the balloon (2). The shock wave generator (5) is arranged on the section of the inner tube (33) that is within the balloon (2).

In the embodiment 4, the electrode ring (51) is made from a conductive metal material, preferably 304 stainless steel. The insulating sleeve (52) is made from an insulating material, such as PI (polyimide). The metal sleeve (53) is also made from a conductive metal material, preferably 304 stainless steel. The insulating glue (54) is an insulating UV adhesive, such as Loctite 349 UV glue.

During use, the embodiment 4 is positioned at the tricuspid valve. After introducing a conductive fluid, the balloon body is inflated. Once the tricuspid valve is expanded by the arcuate protrusions (25), the space between the tricuspid valve wall and the arcuate recesses is not completely blocked, allowing blood flow at the blood flow passage (21) formed by the arcuate recesses. Upon triggering the shock wave generator (5), shock wave energy is released around, thereby shattering the calcified portions and restoring the physiological function of the tricuspid valve. This approach allows for the repair of the tricuspid valve without the need for an artificial valve, and the blood flow is not completely blocked during the repair process, enhancing the safety of the procedure.

### Embodiment 5

The embodiment 5 discloses a shock wave balloon dilation catheter, which adopts the design of the shock wave balloon dilation catheter described in the embodiment 1, thus, details are not reiterated here.

### Embodiment 6

The embodiment 6 discloses a shock wave balloon dilation catheter, which adopts the design of the shock wave balloon dilation catheter described in the embodiment 2, thus, details are not reiterated here.

### Embodiment 7

The embodiment 7 discloses a shock wave balloon dilation catheter, which adopts the design of the shock wave balloon dilation catheter described in the embodiment 3, thus, details are not reiterated here.

### Embodiment 8

The embodiment 8 discloses a shock wave balloon dilation catheter, which adopts the design of the shock wave balloon dilation catheter described in the embodiment 4, thus, details are not reiterated here.

1: A shock wave balloon dilation catheter, which sequentially comprises, from the distal end to the proximal end, a balloon (2) and a dilation catheter (3), characterized in that:
said balloon (2) is provided with a blood flow passage (21) on the outer circumference and/or inside of said balloon (2), said blood flow passage (21) extends axially along said balloon (2) and penetrates through the outer circumference and/or the center of said balloon (2), a shock wave generator (5) is provided in the chamber of said balloon (2), and conductive fluid can be introduced into the chamber of said balloon (2);
the distal end of said dilation catheter (3) either passes through said balloon (2) or enters into said balloon (2), said dilation catheter (3) is sealedly connected to said balloon (2), a fluid passage (32) is provided on said dilation catheter (3) that communicates with the chamber of said balloon (2), said shock wave generator (5) is fixed to the portion of said dilation catheter (3) that is positioned within the chamber of said balloon (2), and said dilation catheter (3) is provided with a guide wire lumen (31).

2: The shock wave balloon dilation catheter, as recited in claim 1, characterized in that: said balloon (2) is composed of at least three balloon bodies (23), supporting rods (24) are provided at both the proximal end and the distal end of said balloon bodies (23), respectively, said support rods (24) at the proximal end of said balloon bodies (23) are formed by branches from the wall of said dilation catheter (3), the distal end of said balloon body (23) is a sealed surface, said support rods (24) at the proximal end and the distal end of said balloon bodies (23) are fixedly connected to said dilation catheter (3), respectively, said dilation catheter (3) passes through the center of said balloon (2), said shock wave generator (5) is provided in the chamber of at least two of said balloon bodies (23), and said balloon body (23) with said shock wave generator (5) is provided with a fluid-conducting tube (22), said shock wave generator (5) is fixed onto said fluid-conducting tube (22), the distal end of said fluid-conducting tube (22) is fixed to the distal end of the chamber of said balloon body (23), and the proximal end of said fluid-conducting tube (22) is fixed to the proximal end of the chamber of said balloon body (23), said support rod (24) at the proximal end is provided with support rod cavity (241) that communicates with the chamber of said balloon body (23) and said fluid passage (32), the proximal end of said fluid-conducting tube (22) in the balloon body (23) with said shock wave generator (5) communicates with said support rod cavity (241), and said fluid-conducting tube (22) is provided with through-holes (221).

3: The shock wave balloon dilation catheter, as recited in claim 2, characterized in that: the number of said balloon bodies is a multiple of 2.

4: The shock wave balloon dilation catheter, as recited in claim 1, characterized in that: the outer wall of said balloon (2) is provided with at least three arcuate protrusions (25) distributed circumferentially along said balloon (2), the inner cavity of said arcuate protrusions (25) communicates with the inner cavity of said balloon (2), forming an integral structure, an arcuate recess is provided between said two adjacent arcuate protrusions (25), forming a blood flow passage (21), the distal end of said dilation catheter (3) passes through the chamber of said balloon (2), while the proximal end and the distal end of said balloon (2) are sealedly connected to said dilation catheter (3).

5: The shock wave balloon dilation catheter, as recited in claim 4, characterized in that: reinforcing ribs (26) are provided on said arcuate recesses, said reinforcing ribs (26) are arranged axially along said balloon (2).

6: The shock wave balloon dilation catheter, as recited in claim 1, characterized in that: said shock wave generator (5) comprises a plurality of electrode rings (51), insulating sleeves (52), and metal sleeves (53), the number of said insulating sleeves (52) is the same as the number of said electrode rings (51), said dilation catheter (3) is provided with conducting wires (6), said conducting wires (6) are provided with exposed copper sections, said metal sleeves (53) are fitted over the exposed copper sections of said conducting wires (6), said insulating sleeves (52) are arranged outside of said metal sleeves (53) to cover said metal sleeves (53), said electrode rings (51) are fitted over said insulating sleeves (52), at least one electrode ring discharge through-hole (511) is arranged on said electrode rings (51), and said electrode ring discharge through-holes (511) are oriented toward the outer circumference of said balloon (2), said insulating sleeve through-holes (512) that expose said metal sleeves (53) are provided at the location where said insulating sleeves (52) are located in said metal sleeves (53).

7: The shock wave balloon dilation catheter, as recited in claim 6, characterized in that: said two adjacent electrode ring discharge through-holes (511) on said electrode rings (51) are arranged in a staggered manner.

8: The shock wave balloon dilation catheter, as recited in claim 7, characterized in that: said electrode rings (51) consist of two symmetrically arranged ring bodies (512), a connecting portion (513) is provided between said two ring bodies (512), and said two ring bodies (512) are connected by said connecting portion (513), said electrode ring discharge through-holes (522) are respectively arranged on said two ring bodies (512).

9: The shock wave balloon dilation catheter, as recited in claim 8, characterized in that: a plurality of said electrode rings (51) are connected in series or parallel by said conducting wires (6).

10: The shock wave balloon dilation catheter, as recited in claim 9, characterized in that: when a plurality of said electrode rings (51) are connected in series by said conducting wires (6), said conducting wires (6) comprise at least one positive wires (61) and a common negative wire (62), said positive wires (61) are provided with exposed copper sections at said electrode rings discharge through-holes (511) of each of said electrode rings (51); or
said conducting wires (6) further comprise a connecting wire (63), the exposed copper section of said positive wire (61) is aligned with said electrode ring discharge through-hole (511) of said electrode ring (51) of the nearest end, both ends of said connecting wire (63) are provided with exposed copper sections, the exposed copper sections of said connecting wire (63) are aligned with said electrode ring discharge through-hole (511) of one of said two adjacent electrode rings (51).

11: The shock wave balloon dilation catheter, as recited in claim 9, characterized in that: when a plurality of said electrode rings (51) are connected in parallel by said conducting wires (6), said conducting wires (6) comprise positive wires (61) and negative wires (62) that match the number of said electrode rings (51), the exposed copper section of each of said positive wires (61) corresponds to one of said electrode ring discharge through-hole (511) of said one electrode ring (51), and the exposed copper section of each of said negative wires (62) corresponds to another of said discharge through-hole of said electrode ring (51).

12: The shock wave balloon dilation catheter, as recited in claim 1, characterized in that: said shock wave generator (5) comprises at least one pair of electrode plates (55), a gap is arranged between each pair of said electrode plates (55), said dilation catheter (3) is provided with said conducting wires (6), said conducting wires (6) are electrically connected to said electrode plates (55).

13: The shock wave balloon dilation catheter, as recited in claim 12, characterized in that: said electrode plates (55) are sheet-shaped.

14: The shock wave balloon dilation catheter, as recited in claim 13, characterized in that: each pair of said electrode plates (55) is connected in series or parallel by said conducting wires (6).

15: The shock wave balloon dilation catheter, as recited in claim 14, characterized in that: when each of said electrode plates (55) pairs is connected in series by said conducting wires (6), said conducting wires (6) comprise a positive wire (61), a connecting wire (63) and a negative wire (62), one of said electrode plate (55) in a pair is electrically connected to said positive wire (61), the other said electrode plate (55) in the same pair is electrically connected to one of said electrode plate (55) in another pair through said connecting wire (63), and the remaining of said electrode plate (55) in that second pair is electrically connected to said negative wire.

16: The shock wave balloon dilation catheter, as recited in claim 14, characterized in that: when each of said electrode plates (55) pairs is connected in parallel by said conducting wires (6), at least two sets of said conducting wires (6) are provided, with each set comprising a positive wire (61) and a negative wire (62), one pair of said electrode plates (55) is electrically connected to one set of said positive wire (61) and said negative wire (62), respectively, while another pair of said electrode plates (55) is electrically connected to another set of said positive wire (61) and said negative wire (62), respectively, forming a parallel connection.

17: A shock wave balloon dilation catheter for the tricuspid valve opening, characterized in that: comprising the shock wave balloon dilation catheter, as recited in claim 1.

18: A shock wave balloon dilation catheter for the tricuspid valve opening, characterized in that: comprising the shock wave balloon dilation catheter, as recited in claim 4.
